(19) United States
(12) Patent Application Publication
(10) Pub. No.: US 2006/0098776 A1
(43) Pub. Date: May 11, 2006

(54) Digital X-ray system

(76) Inventors: Robert Lawrence Burbury, Springfield, MO (US); Matthew Craig Hohenshell, Ozark, MO (US)

Correspondence Address: Wm. Bruce Day;DAY LAW FIRM, P.C., Suite 300, 4330 Belleview, Kansas City, MO, 64111, US

(21) Appl. No.: 10/975,763

(22) Filed: Oct. 28, 2004

(51) Int. Cl. H05G 1/64

(52) U.S. Cl. 378/98.3

(57) ABSTRACT

A digital X-ray machine and operating control system includes and X-ray source and software operated processor control system. The operator may enter patient characteristics affecting radiological density into a control panel so that a processor determines X-ray signal strength. The X-ray beam is collimated, passes through the patient and the support table and then through an ion chamber. A scintillator device converts the X-rays to light in the 600-650 nm range, and passes the light in a straight line from the X-ray source through a beam attenuator in the form of a spectral filter controlling the light transmission. The light then passes through an optical lens gathering the light and finally into a CCD camera. The CCD is sensitive to light in the 600-650 nm range and converts the light to electrical pulses for producing an X-ray image of the patient. Imaging software optimizes the image of the radiograph to diagnostic quality. The system is intended for small animal veterinary use but may be adapted for human use.

### FIELD OF THE INVENTION

This invention relates to X-ray machines and in particular, those machines adapted for veterinary use, although the novel features of the described machine system may be adapted for human usage.

### BACKGROUND OF THE INVENTION

Conventional analog and digital X-ray systems are well known. Digital X-ray systems are becoming favored because of the ease of transmitting an X-ray image to colleagues for consultation, for storage purposes, and for substantially immediate viewing without a need for film development. In some so-called digital systems, a conventional X-ray film is developed and the image scanned, resulting in a GIF of TIF file, which may be transmitted or stored electronically. This system is not a true digital system and requires the normal time involved for film developing, then analog to digital conversion, and image clarity is diminished in the conversion. This conversion system is one which is widely being installed in human health medical centers today, and advertised as “digital” when the system is only partially digital, results in extra work and expense and loses image clarity.

A second type of digital X-ray imagery being used in human health care facilities is a true digital system, in which X-rays from a generator head are passed through a subject, through an ion chamber and scintillator plate which converts the X-rays to light spectrum rays. The light rays are bent at a right angle and enter a charge couple device (CCD) camera, where a digital image is received, composed, an transmitted by the processor circuitry within the camera system. The CCD camera is mounted at a right angle to the X-ray direction from the generator head so as to be out of the line of direct S-ray beam path. Heretofore, the CCD camera was mounted out of the direct X-ray beam path so as to avoid damage to the light receiving sensor components of the CCD. This results in at least some optical distortion, adds cost because of the necessity for high quality optics, and requires additional space to turn the optical path.

Additionally, conventional and digital X-ray systems have utilized a pre-set schedule of X-ray transmission time and intensity. The operator uses a look up table of patient size and weight and sets such parameters into a generator controlled unit by dialing in exposure time and beam height and width as an intensity control. The difficulties of determining settings from a look-up table are exacerbated when an X-ray system is used in a veterinary setting. There, small animals of different species have different radiological densities because differing muscle mass and bone structure, even when of the same species, such as canine, size and weight can differ substantially between breeds. Known X-ray systems are not understood to provide a simple and error reducing method of adjustment to vary X-ray signal strength nor to provide a feed back to optimize the image by controlling X-ray time and energy.

### OBJECTS OF THE INVENTION

The objects of the present invention are:

To provide a digital X-ray system adapted for veterinary use;

To provide such a digital X-ray system in which X-ray beam width and height can be set from a range of predetermined sizes;

To provide such a digital X-ray system wherein the X-ray generator is computer controlled to achieve an optimum image;

To provide such a digital X-ray system which is compact and optically efficient; and

To provide such a digital X-ray system which is well-suited to the task.

Other objects and advantages of the invention will be apparent from the following detailed description.

### SUMMARY OF THE INVENTION

The present invention is a digital X-ray system which is adapted for veterinary use, although aspects of the invention are fully transferable to human X-ray systems. The system includes a programmable controller for the X-ray generator and has feedback circuitry with software for optimizing the digital X-ray image.

The image is recorded by a CCD camera which is in line with the X-ray generator for minimal loss of image clarity. The system provides real time transmission to interested viewers and for digital storage without loss of resolution and time caused by analog to digital conversions.

The system is adaptable for large and small companion animals. Operating selection inputs control X-ray generator emissions to accommodate radiological density and size characteristics of common companion animals.

### BRIEF DESCRIPTION OF THE DRAWINGS

FIG. 1 is a elevational view of the digital X-ray machine and system for veterinarian use.

FIG. 2 is a diagrammatic representation of the system.

FIG. 3 is a longitudinal sectional view of an X-ray receiver component assembly.

FIG. 4 is a system control flow chart.

### DESCRIPTION OF THE PREFERRED AND ALTERNATE EMBODIMENTS

A digital X-ray system is provided which enables true digital radiography in which the X-ray image is captured by a CCD camera, processed to optimize the image and the image presented for immediate viewing and digital storage. The disclosed digital X-ray system is particularly adapted for veterinarian use and provides computer controlled inputs selected for companion animals. Companion animals are those defined as household pets; these include canines, felines, and exotics such as birds, reptiles, and rodents. The system could be adapted for equine, bovine and zoo species. Indeed, certain aspects of this system may be adapted to human use and the following disclosure and claims are not limited to a particular species or field of use unless other wise stated.

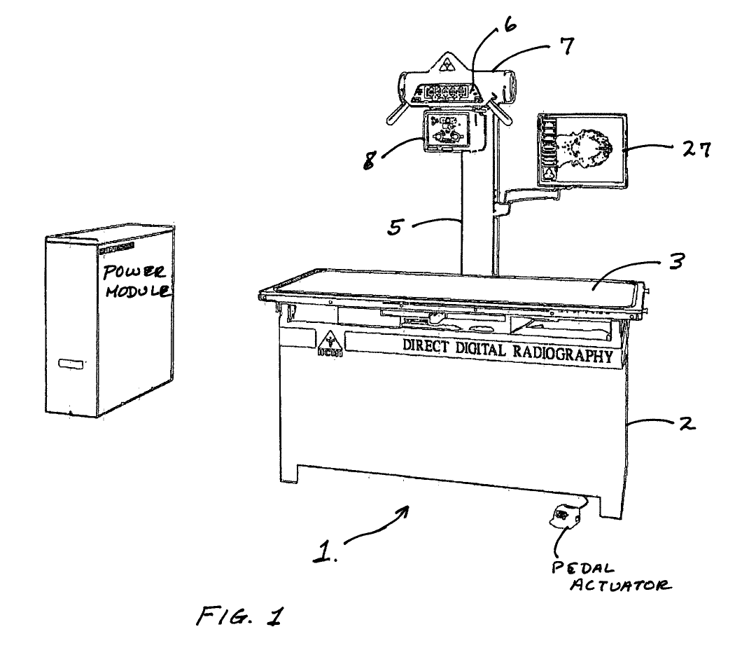

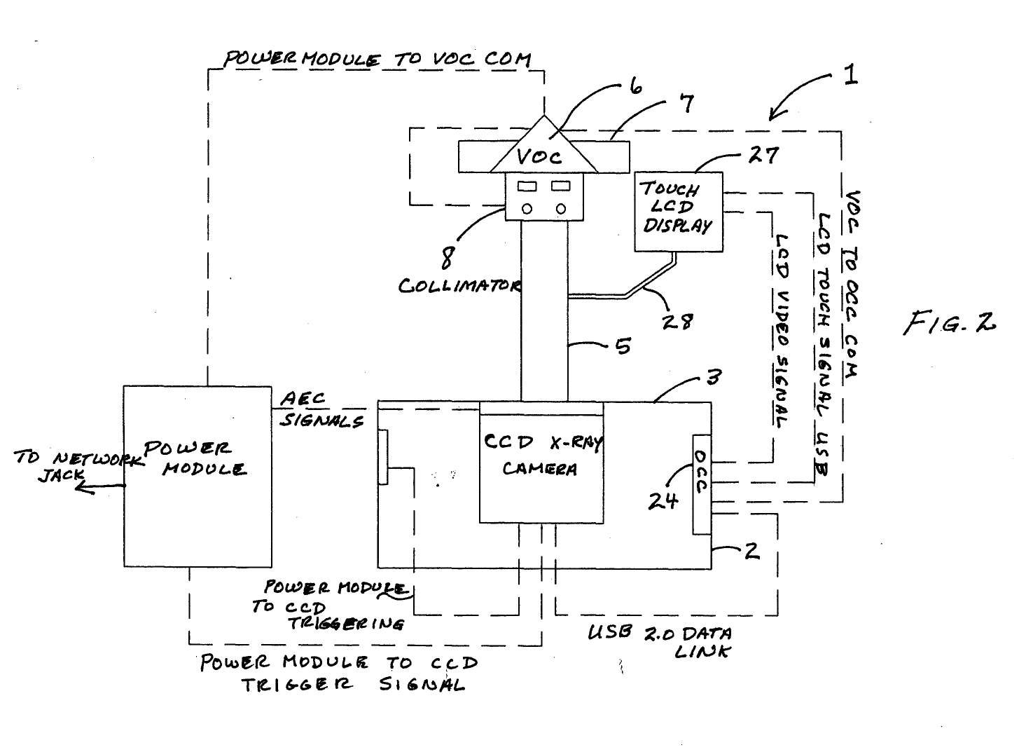

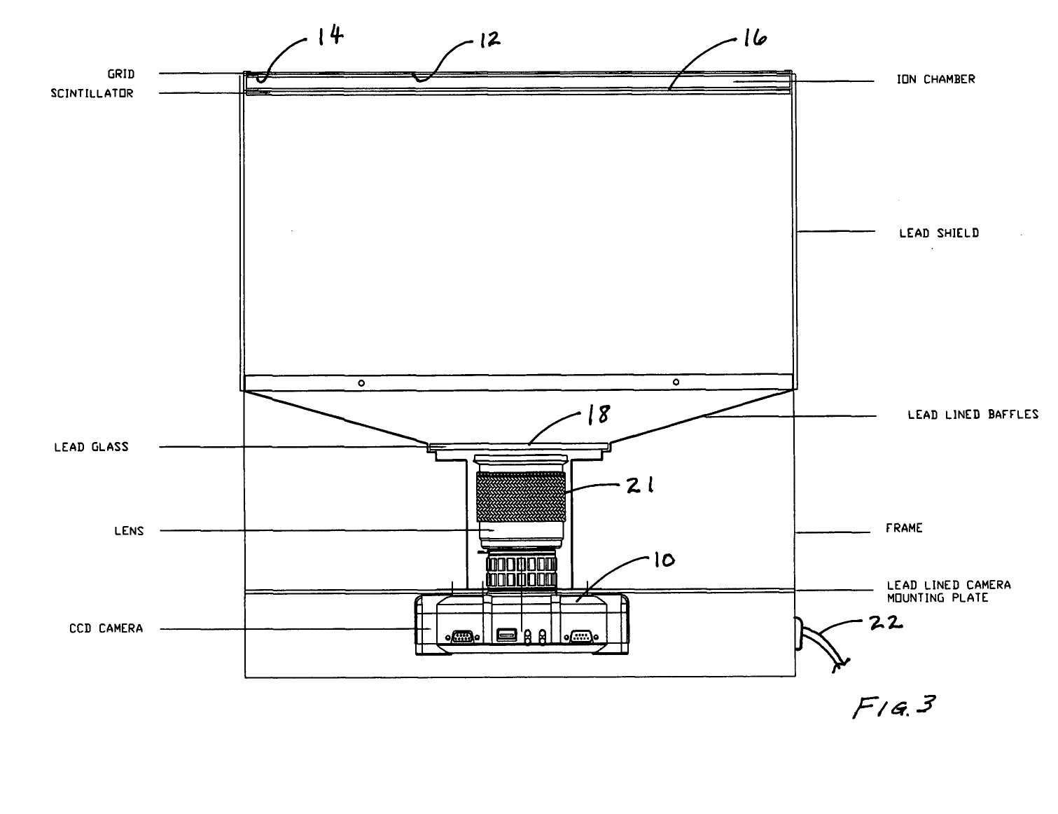

A digital X-ray system, generally indicated as numeral 1, FIGS. 1 and 2, consists of several major components electrically connected together and controlled by a computer system. The mechanical components of the system commence with a base cabinet 2 on which is mounted a patient table 3. The patient table 3 is adapted for supporting various species and sizes of companion animals. The patient supporting t able is selected in size and dimensions for veterinary practice companion animals, however, if the digital X-ray system 1 is further adapted for sue with other animals species, the patient support table 3 is a readily changeable feature. The support table 3 is of a radiologically transparent material and is fabricated to veterinary standards.

An upstanding support beam 5 extends upwardly from the base cabinet 2 and supports at its upper end a veterinary operator control (VOC) 6 positioned against an X-ray generator 7. The generator 7 passes an X-ray beam through a collimator 8 and through a subject positioned on the support table 3. An imaging receptor assembly 10 is located beneath the patient support table 3 within the base cabinet 2 and receives, collects and processes the image data.

In greater detail, the preferred X-ray generator 7 is capable of emitting a pulse width modulated beam of 200 KHz with a constant potential of 37.5 KW. A veterinary operator control panel 6 provides for several inputs and indicators, such as through a touch-screen panel. The inputs provide a set-up for exposure and exposure technique. The first selection possible is “animal species” and “weight” range. Animal species selections are possible between “feline”, “canine” or “exotic” (birds, snakes and turtles). “Weights” are classified in ranges with four ranges per category; small, medium, large and extra large. Selecting a species and weight range automatically sets X-ray exposure start parameters in the X-ray generator 7 in kV mA and density for the X-ray exposure. Communications from inputs at the operator's control sends data to system computer to configure a computer control device (CCD) camera located within the imaging receptor assembly 10. The sent data is also used to select imaging processing filters.

Field of view (FOV) is selected in one of four sizes, including 8×10, 10×12, 11×14 and 14×17 inches. The FOV is pre-selected into the system software from the operator's control console and transmitted via can-bus data link to the X-ray beam collimator 8. Within the collimator 8, a set of motor driven shutters collimate the radiation area to one of the four FOV sizes. The FOV size is also used to crop the image data file size so that only data in the active FOV image area is used for processing. This reduces the file size and the needed image processing time. Making the exposure only requires two selections, species and weight. All camera and computer setups start with an exposure preparation period, which takes approximately two seconds. An audible indicator informs the operator that the system is “ready to expose” and a ready light on the monitor verifies “preparation ready.”

With the transmission of an X-ray from the generator 7 to the collimator 8, the rays pass through the subject of the X-ray and into the imaging receptor assembly 10. The receptor assembly 10 includes an upper grid 12 underlain by an ion chamber 14 which is an X-ray to electrical signal converter and then into a scintillator plate 16. The scintillator plate 16 is preferably a rare-earth phosphor coated plate which converts the X-ray photons to light photons in the 600-650 nanometer (nm) range. Continuing downwardly and into the receptor assembly 10, any X-rays passing through the scintillator plate 16 are absorbed by lead shielding in the interior of the assembly box, or absorbed by a Shott glass filter 18 limiting the rays to the 600-650 nanometer range. Absorption by the filter 18 is intended to be so complete that no X-rays are able to pass through the receptor assembly 10 and into a camera assembly 20 located at the bottom of the receptor box 10 because any X-rays that pass directly into the camera would harm the image. The camera is a CCD (charge coupled device) camera sensitive to light in the 600-650 nanometer range having an optical glass lens 21. Cables 22 connect the CCD camera 20 to a system computer. The CCD camera assembly 20 is cooled by appropriate air flow vents.

Referring to FIG. 2, the base container 2 contains an operator controlled computer 24. Inputs to the operator controlled computer 24 are done through the veterinary operator control 6 which broadcasts a data set containing all information about the upcoming exposure to the OCC 24. The data transmission provides information about the technique, exposure time, kVp, mAs tube angle and the field of view. With respect to tube angle, the X-ray generator 7 is swivel-mounted so that an oblique radiograph may be taken. The OCC receives and stores the exposure date and based upon the values transmits the necessary camera controls internal functions. The OCC also sets the collimator 8 for field of view size and sets exposure values for the X-ray generator 7. The X-ray CCD camera then readies and waits for the exposure triggering signal from the X-ray power module.

Upon exposure, the VOC begins expose signals to the X-ray power module. The X-ray power module then sends a begin expose signal to the X-ray CCD camera 20 and waits for a resulting signal that the camera is ready. Then both the X-ray power module and the X-ray CCD camera simultaneously begin an exposure. The ion chamber 14 senses the presence of X-rays and broadcasts a signal to the X-ray generator 7 for the purpose of controlling radiation. Immediately after exposure has finished, a final data set is transmitted from the VOC to the OCC. This data confirms the initial preparation data set and includes the computer final dosage value. Based upon this data, the OCC ten requests the image data from the X-ray CCD camera. The image data is then transferred via a USB 2.0 from the camera to an area of memory in the OCC. The OCC then begins to process the image data using various filters, algorithms, processes, and cropping. The amount of processing and type is determined by the data transmitted by the VOC about that exposure. After all imaging processing has been completed, the image is displayed on an LCD touch image display 27 mounted on an arm 28 extending outwardly from the upstanding support beam 5.

During the X-ray exposure, the ion chamber 14 directly receives central radiation corresponding directly to the same radiation quantity that contacts the scintillation layer 16 in the receptor assembly. The conversion from radiation to electrical signals takes place in the ion chamber circuit. The ion chamber's signal is amplified an sent to the X-ray power module system controller where the integration of the signal is compared to a look-up table built into software of the operator control computer 24. If the level of radiation contacting the ion chamber is at a satisfactory level, the radiation level being emitted is not changed and is allowed to reach a trip point to end the exposure. If the radiation level is too low, the adjustment of X-ray tube filament power is changed to increase the radiation emitted which results in racing the trip point faster an shortening the exposure time.

At the end of the X-ray exposure a computed milliamp second time is displayed and communicated to the system computer for posting on the image file.

The foregoing is intended to be representative of the invention which may be embodied in various forms and with the use of various means and devices. The particular form disclosed is exemplary only and is not to be taken as limiting except in so far as set forth in the following claims.

What is claimed and desired to be secured by Letters Patent is:

1. A digital X-ray machine assembly comprising:
a) a table for supporting a patient;
b) an X-ray source and control mounted in spaced relationship to said table and emitting X-rays to the following, arrayed in a direct beam path line from said X-ray source;
c) an X-ray beam limiting collimator;
d) an ion chamber positioned below said table;
e) a scintillator plate device converting X-rays to light in the 600-650 nanometer range;
f) an X-ray beam attenuator blocking passage of substantially all X-rays;
g) a spectral filter controlling transmission of light through the filter to that in the 600-650 nanometer range;
h) an optical lens; and
i) a CCD sensitive to light in the 600-650 nanometer range and converting light to electrical impulses for producing an X-ray image of said patient.

2. A digital X-ray machine and software operating system comprising:
a) a table for supporting a patient;
b) an X-ray source and control mounted in spaced relationship to said table and emitting X-rays to the following components, arrayed in a direct beam path line from said X-ray source, the control incorporating a software operating system and providing
i) a plurality of operator pre-sets in which the operator enters patient anatomy characteristics and weight range;
ii) the operator initiating an X-ray exposure sequence;
iii) the system software selecting an X-ray beam width and height from a range of predetermined sizes;
iv) the system software controlling a CCD signal strength and pixel binning to set up a CCD camera;
v) the system software sending an exposure start signal to said CCD;
vi) the CCD sending an exposure command signal to start said X-ray source;
vii) an ion chamber in the X-ray beam path sampling X-ray radiation proportionally and converting X-rays to electrical signals;
viii) the system software analyzing the signal from the ion chamber and adjusting the time and energy of the X-ray emission in real time to minimize exposure time;
ix) the CCD transmitting image signal data to an image processor; and
x) the image processor manipulating the image data based upon time and energy to produce an optimum image.

3. A digital X-ray machine with software operating system comprising:
a) a table for supporting a patient;
b) an X-ray source and control mounted in spaced relationship to said table and emitting X-rays to a CCD camera through a scintillator plate, arrayed in a direct beam path line from said X-ray source, the control incorporating a software operating system and providing;
i) a plurality of operator pre-sets in which the operator enters patient anatomy characteristics and weight range;
ii) the operator initiating an X-ray exposure sequence;
iii) the system software selecting X-ray beam width and height from a range of predetermined sizes;
iv) the system software controlling a CCD signal strength and pixel binning to set up a CCD camera;
v) the system software sending an exposure start signal to said CCD camera;
vi) the CCD camera sending an exposure command signal to start said X-ray source; and
vii) the CCD camera actively sampling light received from the scintillator plate to control energy and time of exposure to optimize fill data of the CCD.

4. In a digital X-ray device having an X-ray generator and an ion chamber converting X-ray energy to electrical signals, an improved image receptor assembly comprising:
a) a scintillator layer primarily emitting light in the 600-650 nm range;
b) a optically transparent X-ray attenuator;
c) A spectral band pass filter, filtering out all light except the 600-650 m range;
d) an optical lens reducing image size; and
e) a CCD converting received light received in the 600-650 nm range to electrical signals.

5. In a digital X-ray device having an X-ray generator, an image receptor assembly comprising:
a) a scintillator layer primarily emitting light in the 600-650 nm range;
b) an optically transparent X-ray attenuator;
c) a spectral band pass filter filtering all light except the 600-650 nm wavelength;
d) an optical lens reducing image size; and
e) a CCD converting received light in the 600-650 nm wavelength to electrical signals, and communicating with said X-ray generator to control parameters of X-ray transmission.

6. A software system providing a method of manipulating X-ray image data comprising of the steps of:
a) automatically analyzing image data based on pre-set information including patient parameters, energy of X-ray signal and angle of X-ray transmission other than right angle to the patient;
b) determining the proper process manipulation of the image data to optimize the resultant image quality; and
c) displaying the image.

7. In a digital X-ray system, an improved camera comprising: a shutterless camera body having a lens gathering light produces by X-rays converted to light, and electronic controls in the camera body controlling and synchronizing X-ray emissions to operation of the camera.

8. The improved camera set forth in claim 7 wherein said lens has a spectral filter limiting optical transmission to 600-650 nm.

9. The improved camera set forth in claim 7 wherein said lens is characterized by the absence of a controllable aperture and has X-ray attenuating elements.

10. The improved camera set forth in claim 7 wherein said lens is zoom lens.

11. The improved camera set forth in claim 7 wherein said electronic controls detect light intensity and convert light to electrical signals representative of light quantity.

12. An X-ray generator apparatus for a digital radiography system comprising:
a) an operator control panel;
b) a control system receiving input from said control panel of patient parameters including body weight;
c) an X-ray generator having variable power outputs;
d) an X-ray beam collimator limiting X-ray field size; and
e) the control system communicating with a camera receiving image signals produced form said X-rays and controlling operation of said X-ray generator based upon time and energy of said X-rays to minimize exposure time and optimize image quality.
